(19) United States
(12) Patent Application Publication
(10) Pub. No.: US 2002/0081635 A1
(43) Pub. Date: Jun. 27, 2002

(54) Novel antibody compositions for preparing enriched T cell preparations

(76) Inventors: Terry E. Thomas, Vancouver (CA); Carolyn Horrocks, Stockholm (SE); Maureen Fairhurst, Vancouver (CA)

Correspondence Address: KILPATRICK STOCKTON LLP, Attn: John S. Pratt, Esq., Suite 2800, 1100 Peachtree Street, Atlanta, GA, 30309-4530, US

(21) Appl. No.: 09/853,881

(22) Filed: May 11, 2001

Related U.S. Application Data
(60) Provisional application No. 60/203,480, filed on May 11, 2000.

(51) Int. Cl. A61K 39/395; G01N 33/567; C07K 16/28

(52) U.S. Cl. 435/7.21; 424/144.1; 530/388.7

(57) ABSTRACT

The present invention relates to antibody compositions and methods that are useful in preparing suspensions enriched for T cell subsets. The invention also relates to kits for carrying out the processes and to the cell preparations prepared by the processes.

This application claims the benefit under 35 USC §119(e) from U.S. Provisional Patent Application No. 60/203,480 filed May 11, 2000.

### FIELD OF THE INVENTION

The present invention relates to novel antibody compositions, and processes and kits for preparing enriched T cell preparations.

### BACKGROUND OF THE INVENTION

The study of immune response often requires purified suspensions of minor T cell subsets such as αβTCR, γδTCR, TH1, TH2, naïve, resting, and memory T Cells. These T cell subsets may be useful clinically in vaccine production and immunotherapy. Current separation protocols rely on positive selection of these cell types where the desired cells are labelled with antibodies and then isolated. The major limitation of this approach is that the binding of antibodies to certain T cell markers often has striking biological effects such as blocking or activation. For most applications it is important to recover functionally intact or unperturbed T cells. A negative selection method in which the unwanted cells are labelled is much preferable if not crucial. The isolated desired T cell subset remains unaffected by antibody binding. The present invention identifies combinations of antibodies to cell surface markers which can be used to negatively select αβTCR, γδTCR, TH1, TH2, naïve, resting, and memory T Cells.

Bone marrow derived lymphoid progenitors migrate to the thymus where T cell development and education occurs. After this process, mature naïve T cells expressing either the CD4 or CD8 co-receptors, along with CD3 and functional T cell receptor, exit the thymus (Ashton-Rickardt P G, and Tonegawa S. A differential-avidity model for T-cell selection. Immunol Today. 15(8):362-6, 1994). CD8+ T cells recognize antigen in the context of MHC I and acquire cytotoxic activity. In contrast, CD4+ T cells recognize antigen/MHC II complexes on antigen presenting cells (APC) and are activated to secrete cytokines. Upon appropriate stimulation, the T cells gain expression of additional surface markers such as CD69, CD25 and HLA-DR.

Following activation, naïve T cells either die by apoptosis or become memory cells. Naïve T cells express CD45RA (Sprent J. Immunological memory. Curr Opin Immunol. 9(3):371-9, 1997), whereas memory cells express CD45RO. Memory CD4+ T cells may be further subdivided into Th1 and Th2 (and more recently Th3) cells. These subsets differ in the range of cytokines and chemokines they secrete and can be distinguished by their differential expression of receptors for these molecules (Sallusto F, Lanzavecchia A, and Mackay C R. Chemokines and chemokine receptors in T-cell priming and Th1/Th2-mediated responses. 19(12):568-74, 1998). For example, Th1 cells express IL-12 receptor (IL-12R) whereas Th2 cells express IL-4R.

### SUMMARY OF THE INVENTION

The present inventors have developed antibody compositions for use in preparing cell preparations enriched for T cell types including, but not limited to, resting T cells, naïve T cells, memory T cells, γιT cells, αβT cells, CD4⁺ T cells, CD8⁺ T cells, CD4⁺ resting T cells, CD4⁺ naïve T cells, CD4⁺ memory T cells, CD4⁺ αβT cells, CD4⁺ Th1 T cells, CD4⁺ Th2 T cells, CD8⁺ resting T cells, CD8⁺αβ T cells, CD8⁺ naïve T cells and CD8⁺ memory T cells.

To enrich for T cells generally the antibodies in the antibody composition are specific for selected markers associated with non-T cells thereby allowing them to be removed from the cell preparation. In particular, the present inventors have found using a negative selection technique that an antibody composition containing antibodies specific for (a) CD16 and/or CD66b and/or CD11b and/or CD15; (b) CD19 and/or CD20 and/or CD21 and/or CD22 and/or CD24 and/or Ig; and (c) CD14 and/or CD36 gives a cell preparation highly enriched for human T cells. This combination of antibodies is generally referred to “the base T cell composition or cocktail” herein. Optionally, the T cell composition additionally includes antibodies to CD13, CD34, CD33, CD56, IgE, CD41 or glycophorin A.

In a specific embodiment, the composition for enriching for T cells comprises antibodies specific for the antigens CD16, CD19 and CD36 and optionally antibodies specific for the antigens CD56 and glycophorin A. In one embodiment, the T cell composition is for enriching CD4⁺ T cells and additionally comprises antibodies to CD8. In another embodiment, the T cell composition is for enriching CD8⁺ T cells and additionally comprises antibodies to CD4.

In order to enrich for subsets of T cells, additional antibodies are added to the base T cell composition. Specific antibody combinations that can be used to enrich for T cell subsets are listed in Table 1.

The enrichment and recovery of T cells using the antibody compositions of the invention in a negative selection technique has many advantages over conventional positive selection techniques. Highly enriched cell preparations can be obtained using a single step. The cells obtained using the antibody composition of the invention are not labeled or coated with antibodies or modified making them highly suitable for many uses. For example, the isolated T cells can be used in transplantation and other therapeutic uses.

The present invention also relates to a negative selection process for enriching and recovering T cells in a sample comprising (1)reacting the sample with an antibody composition containing antibodies capable of binding to the antigens (a) CD16 and/or CD66b and/or CD11b and/or CD15; (b) CD19 and/or CD20 and/or CD21 and/or CD22 and/or CD24 and/or Ig; and (c) CD14 and/or CD36 under conditions permitting the formation of conjugates between the antibodies and cells in the sample having the antigens (a) CD16 and/or CD66b and/or CD11b and/or CD15; (b) CD19 and/or CD20 and/or CD21 and/or CD22 and/or CD24 and/or Ig; and (c) CD14 and/or CD36 on their surfaces; (2) removing the conjugates; and (3) recovering a cell preparation which is enriched in T cells. Optionally, the T cell composition additionally includes antibodies to CD13, CD34, CD33, CD56, IgE, CD41 or glycophorin A.

The present invention also relates to a kit useful in preparing a cell preparation enriched in T cells comprising antibodies specific for the antigens (a) CD16 and/or CD66b and/or CD11b and/or CD15; (b) CD19 and/or CD20 and/or CD21 and/or CD22 and/or CD24 and/or Ig; and (c) CD14 and/or CD36, and instructions for preparing a cell preparation enriched in T cells. Optionally, the kit additionally includes antibodies to CD13, CD33, CD34, CD56, IgE, CD41 or glycophorin A.

The invention further relates to cell preparations obtained in accordance with the processes of the invention. The invention still further contemplates a method of using the antibody compositions of the invention in negative selection methods to recover a cell preparation which is enriched in T cells.

These and other aspects of the present invention will become evident upon reference to the following detailed description and attached drawings. In addition, reference is made herein to various publications, which are hereby incorporated by reference in their entirety.

### BRIEF DESCRIPTION OF THE DRAWINGS

The invention will now be described with reference to the accompanying drawings, in which:

FIG. 1 is a schematic representation of magnetic cell labeling using tetrameric antibody complexes and colloidal dextran iron.

FIG. 2 is a schematic diagram of a rosette of erythrocytes formed around an unwanted target nucleated cell using tetrameric antibody complexes.

### DETAILED DESCRIPTION OF THE INVENTION

I. ANTIBODY COMPOSITIONS

As hereinbefore mentioned, the invention relates to an antibody compositions for preparing enriched T cell preparations. In one aspect, the antibody composition is for enriching T cells and comprises antibodies specific for the antigens (a) CD16 and/or CD66b and/or CD11b and/or CD15; (b) CD19 and/or CD20 and/or CD21 and/or CD22 and/or CD24 and/or Ig; and (c) CD14 and/or CD36, which are present on the surface of non-T cells. This combination of antibodies is generally referred to herein as “the base T cell composition or cocktail”. Optionally, the T cell composition additionally includes antibodies to CD13, CD33, CD34, CD56, IgE, CD41 or glycophorin A.

In a specific embodiment, the composition for enriching for T cells comprises antibodies specific for the antigens CD16, CD19 and CD36 and optionally antibodies specific for the antigens CD56 and glycophorin A. In one embodiment, the T cell composition is for enriching CD4⁺ T cells and additionally comprises antibodies to CD8. In another embodiment, the T cell composition is for enriching CD8⁺ T cells and additionally comprises antibodies to CD4.

In one embodiment, the antibody composition is for enriching resting T cells and comprises antibodies capable of binding to the antigens: (a) CD16 and/or CD66b and/or CD11b and/or CD15; (b) CD19 and/or CD20 and/or CD21 and/or CD22 and/or CD24 and/or Ig; (c) CD14 and/or CD36; and (d) HLA-DR and/or CD25 and/or CD69 and/or CD27.

In another embodiment, the antibody composition is for enriching γδ T cells and comprises antibodies capable of binding to the antigens: (a) CD16 and/or CD66b and/or CD11b and/or CD15; (b) CD19 and/or CD20 and/or CD21 and/or CD22 and/or CD24 and/or Ig; (c) CD14 and/or CD36; and (d) γδ TCR.

In a further embodiment, the antibody composition is for enriching αβ cells and comprises antibodies capable of binding to the antigens: (a) CD16 and/or CD66b and/or CD11b and/or CD15; (b) CD19 and/or CD20 and/or CD21 and/or CD22 and/or CD24 and/or Ig; (c) CD14 and/or CD36; and (d) γδ TCR.

In another embodiment, the antibody composition is for enriching naive T cells and comprises antibodies capable of binding to the antigens: (a) CD16 and/or CD66b and/or CD11b and/or CD15; (b) CD19 and/or CD20 and/or CD21 and/or CD22 and/or CD24 and/or Ig; (c) CD14 and/or CD36; and (d) CD45RO and/or CD29.

In yet another embodiment, the antibody compositions for enriching memory T cells comprises antibodies capable of binding to the antigens: (a) CD16 and/or CD66b and/or CD11b and/or CD15; (b) CD19 and/or CD20 and/or CD21 and/or CD22 and/or CD24 and/or Ig; (c) CD14 and/or CD36; and (d) CD45RA.

In yet another embodiment, the antibody composition is for enriching naive CD4⁺ T cells and comprises antibodies capable of binding to the antigens: (a) CD16 and/or CD66b and/or CD11b and/or CD15; (b) CD19 and/or CD20 and/or CD21 and/or CD22 and/or CD24 and/or Ig; (c) CD14 and/or CD36; (d) CD8; and (e) CD45RO and/or CD29. Preferably, the antibody composition for enriching naïve CD4⁺ T cells comprises antibodies capable of binding to the antigens CD16, CD19, CD14, CD8, CD45RO and optionally CD56 and/or glycophorin A.

In a further embodiment, the antibody composition is for enriching memory CD4⁺ T cells and comprises antibodies capable of binding to the antigens: (a) CD16 and/or CD66b and/or CD11b and/or CD15; (b) CD19 and/or CD20 and/or CD21 and/or CD22 and/or CD24 and/or Ig; (c) CD14 and/or CD36; (d) CD8 and (e) CD45RA.

In another embodiment, the antibody composition is for enriching resting CD4⁺ T cells and comprises antibodies capable of binding to the antigens: (a) CD16 and/or CD66b and/or CD11b and/or CD15; (b) CD19 and/or CD20 and/or CD21 and/or CD22 and/or CD24 and/or Ig; (c) CD14 and/or CD36; (d) CD8 and (e) HLA-DR and/or CD25 and/or CD69 and/or CD27.

In a further embodiment, the antibody composition is for enriching CD4⁺ αβ T cells and comprises antibodies capable of binding to the antigens: (a) CD16 and/or CD66b and/or CD11b and/or CD15; (b) CD19 and/or CD20 and/or CD21 and/or CD22 and/or CD24 and/or Ig; (c) CD14 and/or CD36; (d) γδ TCR; and (e) CD8.

In yet another embodiment, the antibody composition is for enriching TH1 CD4⁺ T cells and comprises antibodies capable of binding to the antigens: (a) CD16 and/or CD66b and/or CD11b and/or CD15; (b) CD19 and/or CD20 and/or CD21 and/or CD22 and/or CD24 and/or Ig; (c) CD14 and/or CD36; (d) CD8; and (e) CD124.

In another embodiment, the antibody composition is for enriching TH2 CD4⁺ T cells and comprises antibodies capable of binding to the antigens: (a) CD16 and/or CD66b and/or CD11b and/or CD15; (b) CD19 and/or CD20 and/or CD21 and/or CD22 and/or CD24 and/or Ig; (c) CD14 and/or CD36; (d) CD8; and (e) CCR5.

In a further embodiment, the antibody composition is for enriching naive CD8⁺ T cells and comprises antibodies capable of binding to the antigens: (a) CD16 and/or CD66b and/or CD11b and/or CD15; (b) CD19 and/or CD20 and/or CD21 and/or CD22 and/or CD24 and/or Ig; (c) CD14 and/or CD36; (d) CD4; and (e) CD45RO and/or CD29.

In yet another embodiment, the antibody composition is for enriching memory CD8⁺ T cells and comprises antibodies capable of binding to the antigens: (a) CD16 and/or CD66b and/or CD11b and/or CD15; (b) CD19 and/or CD20 and/or CD21 and/or CD22 and/or CD24 and/or Ig; (c) CD14 and/or CD36; (d) CD4; and (e) CD45RA.

In another embodiment, the antibody composition is for enriching resting CD8⁺ αβ T cells and comprises antibodies capable of binding to the antigens: (a) CD16 and/or CD66b and/or CD11b and/or CD15; (b) CD19 and/or CD20 and/or CD21 and/or CD22 and/or CD24 and/or Ig; (c) CD14 and/or CD36; (d) CD4; and (e) HLA-DR and/or CD25 and/or CD69 and/or CD27.

In a further embodiment, the antibody composition is for enriching CD8⁺ αβ T cells and comprises antibodies capable of binding to the antigens: (a) CD16 and/or CD66b and/or CD11b and/or CD15; (b) CD19 and/or CD20 and/or CD21 and/or CD22 and/or CD24 and/or Ig; (c) CD14 and/or CD36; (d) γδ TCR; and (e) CD4.

The above antibody compositions may additionally include other antibodies such antibodies that can bind to the antigens CD13, CD33, CD34, CD56, IgE, CD41 or glycophorin A. Antibodies useful in the invention may be prepared as described below using techniques known in the art or may be obtained from commercial sources as outlined in Table 2.

One skilled in the art will appreciate that in addition to the antibodies listed above and in Table 1, the T cell enrichment cocktail may additionally include other antibodies that are specific for antigens on the surface of cells you wish to deplete from the sample including those listed in Table 2. The selection of the antibodies can depend on many factors including the nature of the sample to be enriched.

Within the context of the present invention, antibodies are understood to include monoclonal antibodies and polyclonal antibodies, antibody fragments (e.g., Fab, and F(ab′)₂) and chimeric antibodies. Antibodies are understood to be reactive against a selected antigen on the surface of a non-T cell if they bind with an appropriate affinity (association constant), e.g. greater than or equal to 10⁷ M⁻¹.

Polyclonal antibodies against selected antigens on the surface of unwanted cells may be readily generated by one of ordinary skill in the art from a variety of warm-blooded animals such as horses, cows, various fowl, rabbits, mice, hamsters, or rats. For example, a mammal, (e.g., a mouse, hamster, or rabbit) can be immunized with an immunogenic form of an antigen which elicits an antibody response in the mammal. Techniques for conferring immunogenicity on an antigen include conjugation to carriers or other techniques well known in the art. For example, the antigen can be administered in the presence of adjuvant. The progress of immunization can be monitored by detection of antibody titers in plasma or serum. Following immunization, antisera can be obtained and polyclonal antibodies isolated from the sera.

Monoclonal antibodies are preferably used in the antibody compositions of the invention. Monoclonal antibodies specific for selected antigens on the surface of unwanted cells may be readily generated using conventional techniques. For example, monoclonal antibodies may be produced by the hybridoma technique originally developed by Kohler and Milstein 1975 (Nature 256, 495-497; see also U.S. Pat. Nos. RE 32,011, 4,902,614, 4,543,439, and 4,411,993 which are incorporated herein by reference; see also Monoclonal Antibodies, Hybridomas: A New Dimension in Biological Analyses, Plenum Press, Kennett, McKearn, and Bechtol (eds.), 1980, and Antibodies: A Laboratory Manual, Harlow and Lane (eds.), Cold Spring Harbor Laboratory Press, 1988). Other techniques may also be utilized to construct monoclonal antibodies (for example, see William D. Huse et al., 1989, “Generation of a Large Combinational Library of the Immunoglobulin Repertoire in Phage Lambda,” Science 246:1275-1281, L. Sastry et al., 1989 “Cloning of the Immunological Repertoire in Escherichia coli for Generation of Monoclonal Catalytic Antibodies: Construction of a Heavy Chain Variable Region-Specific cDNA Library,” Proc Natl. Acad. Sci USA 86:5728-5732; Kozbor et al., 1983 Immunol. Today 4, 72 re the human B-cell hybridoma technique; Cole et al. 1985 Monoclonal Antibodies in Cancer Therapy, Allen R. Bliss, Inc., pages 77-96 re the EBV-hybridoma technique to produce human monoclonal antibodies; and see also Michelle Alting-Mees et al., 1990 “Monoclonal Antibody Expression Libraries: A Rapid Alternative to Hybridomas,” Strategies in Molecular Biology 3:1-9). Hybridoma cells can be screened immunochemically for production of antibodies specifically reactive with an antigen, and monoclonal antibodies can be isolated.

The term “antibody” as used herein is intended to include antibody fragments which are specifically reactive with specific antigens on the surface of unwanted cells. Antibodies can be fragmented using conventional techniques and the fragments screened for utility in the same manner as described above for whole antibodies. For example, F(ab′)₂ fragments can be generated by treating antibody with pepsin. The resulting F(ab′)₂ fragment can be treated to reduce disulfide bridges to produce Fab′ fragments.

The invention also contemplates chimeric antibody derivatives, i.e., antibody molecules that combine a non-human animal variable region and a human constant region. Chimeric antibody molecules can include, for example, the antigen binding domain from an antibody of a mouse, rat, or other species, with human constant regions. A variety of approaches for making chimeric antibodies have been described and can be used to make chimeric antibodies containing the immunoglobulin variable region which recognizes selected antigens on the surface of differentiated cells or tumor cells. See, for example, Morrison et al., 1985; Proc. Natl. Acad. Sci. U.S.A. 81,6851; Takeda et al., 1985, Nature 314:452; Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B.

Antibodies may be selected for use in the antibody compositions of the invention based on their ability to deplete targeted unwanted cells and recover non-targeted cells (i.e. T cells or a particular subset thereof) in magnetic cell separations as more particularly described herein, and in U.S. Pat. No. 5,514,340, which is incorporated in its entirety herein by reference.

II. PROCESS FOR PREPARING ENRICHED T CELL PREPARATIONS

The antibody compositions of the invention may be used to isolate suspensions enriched for particular T cell subsets. In accordance with a process of the invention, a sample is reacted with an antibody composition containing antibodies which are specific for selected antigens on the surface of the unwanted cells to be removed from the sample and not on the T cells to be enriched in the sample, under suitable conditions, conjugates form between the antibodies contained in the antibody composition and the cells in the sample containing the antigens on their surface; and the conjugates are removed to provide a cell preparation enriched in specific cells.

In one aspect the present invention provides a negative selection process for enriching and recovering T cells in a sample comprising (1) reacting the sample with an antibody composition containing antibodies capable of binding to the antigens (a) CD16 and/or CD66b and/or CD11b and/or CD15; (b) CD19 and/or CD20 and/or CD21 and/or CD22 and/or CD24 and/or Ig; and (c) CD14 and/or CD36, under conditions so that conjugates are formed between the antibodies and cells in the sample containing the antigens (a) CD16 and/or CD66b and/or CD11b and/or CD15; (b) CD19 and/or CD20 and/or CD21 and/or CD22 and/or CD24 and/or Ig; and (c) CD14 and/or CD36 on their surfaces; (2) removing the conjugates; and, (3) recovering a cell preparation which is enriched in T cells.

In order to enrich for particular T cell subsets, the antibody composition will additionally include antibodies to antigens on the T cell subsets one wishes to remove from the sample. Examples of antibody compositions useful for enriching for specific T cell subsets are described above under “Antibody Compositions” and are also listed in Table 1.

In the above negative selection processes of the invention for T cell enrichment, conditions which permit the formation of conjugates may be selected having regard to factors such as the nature and amounts of the antibodies in the antibody composition, and the estimated concentration of targeted cells in the sample.

The antibodies in the antibody compositions may be labelled with a marker or they may be conjugated to a matrix. Examples of markers are biotin, which can be removed by avidin bound to a support, and fluorochromes, e.g. fluorescein, which provide for separation using fluorescence activated sorters. Examples of matrices are magnetic beads, which allow for direct magnetic separation (Kernshead 1992), panning surfaces e.g. plates, (Lebkowski, J. S, et al., (1994), J. of Cellular Biochemistry supple. 18b:58), dense particles for density centrifugation (Van Vlasselaer, P., Density Adjusted Cell Sorting (DACS), A Novel Method to Remove Tumor Cells From Peripheral Blood and Bone Marrow StemCell Transplants. (1995) 3rd International Symposium on Recent Advances in Hematopoietic Stem Cell Transplantation-Clinical Progress, New Technologies and Gene Therapy, San Diego, Calif.), dense particles alone (Zwerner et al., Immunol. Meth. 1996 198(2):199-202) adsorption columns (Berenson et al. 1986, Journal of Immunological Methods 91:11-19.), and adsorption membranes. The antibodies may also be joined to a cytotoxic agent such as complement or a cytotoxin, to lyse or kill the targeted non-T cells.

The antibodies in the antibody compositions may be directly or indirectly coupled to a matrix. For example, the antibodies in the compositions of the invention may be chemically bound to the surface of magnetic particles for example, using cyanogen bromide. When the magnetic particles are reacted with a sample, conjugates will form between the magnetic particles with bound antibodies specific for antigens on the surfaces of the non-T cells and the cells having the antigens on their surfaces.

Alternatively, the antibodies may be indirectly conjugated to a matrix using antibodies. For example, a matrix may be coated with a second antibody having specificity for the antibodies in the antibody composition. By way of example, if the antibodies in the antibody composition are mouse IgG antibodies, the second antibody may be rabbit anti-mouse IgG.

The antibodies in the antibody compositions may also be incorporated in antibody reagents which indirectly conjugate to a matrix. Examples of antibody reagents are bispecific antibodies, tetrameric antibody complexes, and biotinylated antibodies.

Bispecific antibodies contain a variable region of an antibody in an antibody composition of the invention, and a variable region specific for at least one antigen on the surface of a matrix. The bispecific antibodies may be prepared by forming hybrid hybridomas. The hybrid hybridomas may be prepared using the procedures known in the art such as those disclosed in Staerz & Bevan, (1986, PNAS (USA) 83: 1453) and Staerz & Bevan, (1986, Immunology Today, 7:241). Bispecific antibodies may also be constructed by chemical means using procedures such as those described by Staerz et al., (1985, Nature, 314:628) and Perez et al., (1985 Nature 316:354), or by expression of recombinant immunoglobulin gene constructs.

A tetrameric immunological complex may be prepared by mixing a first monoclonal antibody which is capable of binding to at least one antigen on the surface of a matrix, and a second monoclonal antibody from the antibody composition of the invention. The first and second monoclonal antibody are from a first animal species. The first and second antibody are reacted with an about equimolar amount of monoclonal antibodies of a second animal species which are directed against the Fc-fragments of the antibodies of the first animal species. The first and second antibody may also be reacted with an about equimolar amount of the F(ab′)₂ fragments of monoclonal antibodies of a second animal species which are directed against the Fc-fragments of the antibodies of the first animal species. (See U.S. Pat. No. 4,868,109 to Lansdorp, which is incorporated herein by reference for a description of tetrameric antibody complexes and methods for preparing same).

The antibodies of the invention may be biotinylated and indirectly conjugated to a matrix which is labelled with (strept) avidin. For example, biotinylated antibodies contained in the antibody composition of the invention may be used in combination with magnetic iron-dextran particles that are covalently labelled with (strept) avidin (Miltenyi, S. et al., Cytometry 11:231, 1990). Many alternative indirect ways to specifically cross-link the antibodies in the antibody composition and matrices would also be apparent to those skilled in the art.

In an embodiment of the invention, the cell conjugates are removed by magnetic separation using magnetic particles. Suitable magnetic particles include particles in ferrofluids and other colloidal magnetic solutions. “Ferrofluid” refers to a colloidal solution containing particles consisting of a magnetic core, such as magnetite (Fe₃O₄) coated or embedded in material that prevents the crystals from interacting. Examples of such materials include proteins, such as ferritin, polysaccharides, such as dextrans, or synthetic polymers such as sulfonated polystyrene cross-linked with divinylbenzene. The core portion is generally too small to hold a permanent magnetic field. The ferrofluids become magnetized when placed in a magnetic field. Examples of ferrofluids and methods for preparing them are described by Kemshead J. T. (1992) in J. Hematotherapy, 1:35-44, at pages 36 to 39, and Ziolo et al. Science (1994) 257:219 which are incorporated herein by reference. Colloidal particles of dextran-iron complex are preferably used in the process of the invention. (See Molday, R. S. and McKenzie, L. L. FEBS Lett. 170:232, 1984; Miltenyi et al., Cytometry 11:231, 1990; and Molday, R. S. and MacKenzie, D., J. Immunol. Methods 52:353, 1982; Thomas et al., J. Hematother. 2:297 (1993); and U.S. Pat. No. 4,452,733, which are each incorporated herein by reference).

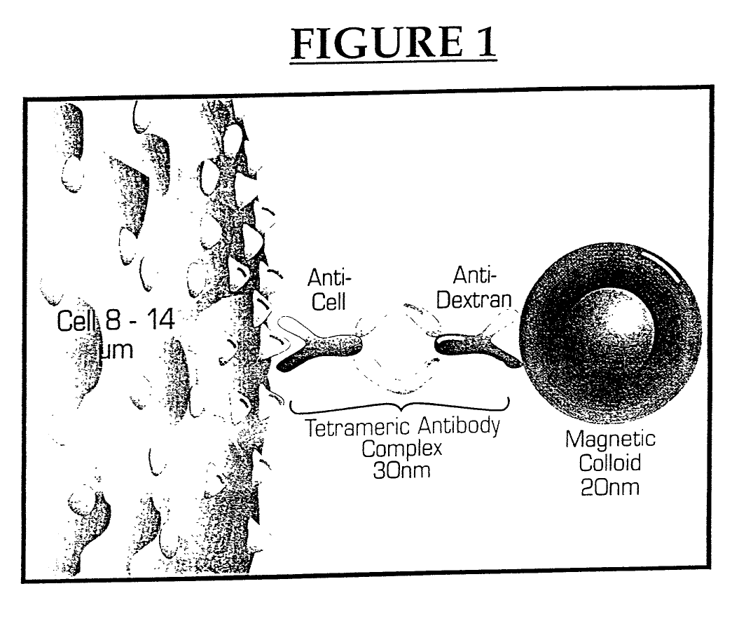

FIG. 1 is a schematic representation of magnetic cell labeling using tetrameric antibody complexes and colloidal dextran iron.

In accordance with the magnetic separation method, the sample containing the T cells to be recovered, is reacted with the above described antibody reagents, preferably tetrameric antibody complexes, so that the antibody reagents bind to the non-T cells present in the sample to form cell conjugates of the targeted non-T cells and the antibody reagents. The reaction conditions are selected to provide the desired level of binding of the targeted non-T cells and the antibody reagents. Preferably the sample is incubated with the antibody reagents for a period of 5 to 60 minutes at either 4° or ambient room temperature. The concentration of the antibody reagents is selected depending on the estimated concentration of the targeted differentiated cells in the sample. Generally, the concentration is between about 0.1 to 50 μg/ml of sample. The magnetic particles are then added and the mixture is incubated for a period of about 5 minutes to 30 minutes at the selected temperature. The sample is then ready to be separated over a magnetic filter device. Preferably, the magnetic separation procedure is carried out using the magnetic filter and methods described in U.S. Pat. No. 5,514,340 to Lansdorp and Thomas which is incorporated in its entirety herein by reference.

The sample containing the magnetically labelled cell conjugates is passed through the magnetic filter in the presence of a magnetic field. In a preferred embodiment of the invention, the magnet is a dipole magnet with a gap varying from 0.3 to 3.0 inches bore and having a magnetic field of 0.5-2 Tesla. The magnetically labelled cell conjugates are retained in the high gradient magnetic column and the materials which are not magnetically labelled flow through the column after washing with a buffer.

The preparation containing non-magnetically labelled cells may be analyzed using procedures such as flow cytometry. The activity of the cells in the preparation may also be assessed for example by measuring T cell proliferation or cytotoxicity in standard assays.

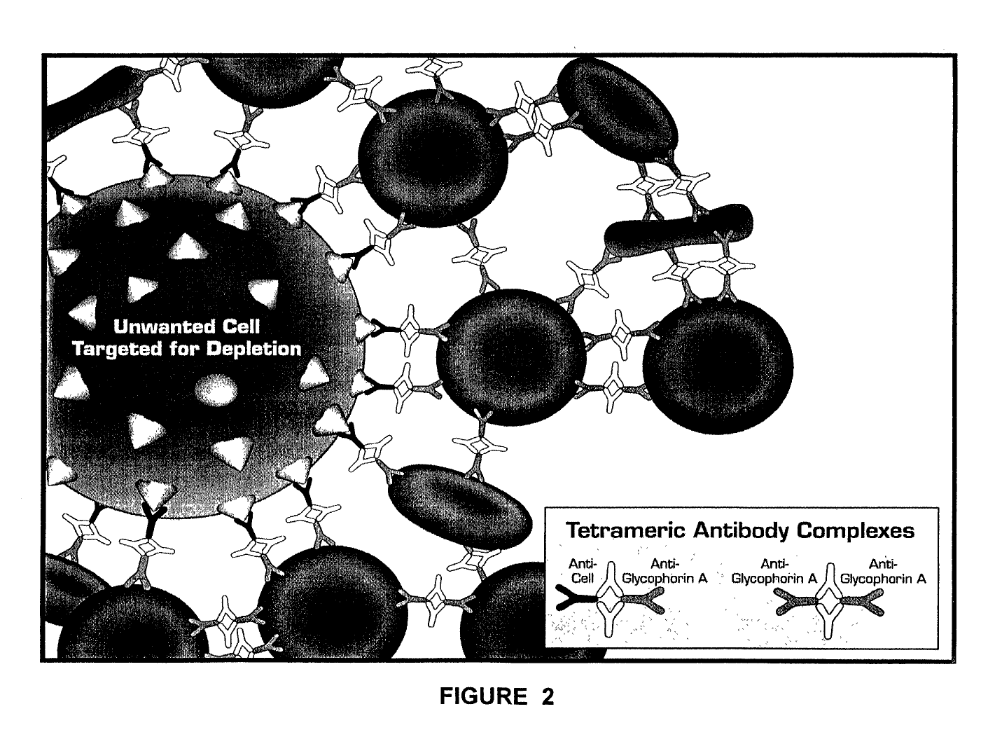

The antibody compositions of the invention may also be used in immunorosetting protocols when the sample to be enriched contains red blood cells. In such an embodiment the undesired cells in the sample are rosetted with the red blood cells by including an antibody that binds to red blood cells (or erythrocytes) in the antibody composition. A suitable antibody is one that binds glycophorin A. FIG. 2 is a schematic diagram showing a rosette of erythrocytes formed around an unwanted target nucleated cell using tetrameric antibody complexes.

Accordingly, the present invention provides a negative selection method for enriching and recovering T cells in a sample containing the T cells, erythrocytes and undesired cells comprising:

(1) contacting the sample with an antibody composition comprising (a) at least one antibody that binds to an antigen on the undesired cells linked, either directly or indirectly, to (b) at least one antibody that binds to the erythrocytes, under conditions to allow immunorosettes of the undesired cells and the erythrocytes to form; and

(2) separating the immunorosettes from the remainder of the sample to obtain a sample enriched in T cells.

The immunorosettes between the erythrocytes and the unwanted cells formed in step (1) can be separated from the desired cells using a variety of techniques. In one embodiment, the sample, containing the immunorosettes, is layered over a buoyant density solution (such as Ficoll-Hypaque) and centrifuged. The immunorosettes pellet and the desired T cells remain at the interface between the buoyant density solution and the sample. The desired cells are then removed from the interface for further use. In another embodiment, the sample containing the immunorosettes obtained in step (1) is mixed with a sedimentation reagent (such as hydroxyethyl starch, gelatin or methyl cellulose) and the rosettes are permitted to sediment. The desired cells remain in suspension and are removed for further use. In a further embodiment, the sample containing the immunorosettes obtained in step (1) is allowed to sediment with or without the aid of centrifugation or Counter Flow Elutriation. The T cells remain in suspension and are removed for further use.

The immunorosetting method may be used in the processing of biological samples that contain erythrocytes including blood (in particular, cord blood and whole blood) bone marrow, fetal liver, buffy coat suspensions, pleural and peritoneal effusions and suspensions of thymocytes and splenocytes. Surprisingly, the inventors have found that the method can be used to remove cells directly from whole blood or whole bone marrow without prior processing. This offers a significant advantage of the method of the invention over the prior art methods. In particular, the erythrocytes do not have to be removed, labelled and added back to the sample.

The antibody compositions for use in step (a) of the immunorosetting method are previously described herein. For example, in an immunorosetting protocol, the T cell composition would comprise antibodies specific for (a) CD16 and/or CD66b and/or CD11b and/or CD15; (b) CD19 and/or CD20 and/or CD21 and/or CD22 and/or CD24 and/or Ig; (c) CD14 and/or CD36; and (d) glycophorin A.

III. KITS

The present invention also includes kits useful in preparing the above described enriched T cell preparations. The kit will comprise an antibody composition useful in enriching a sample for a particular T cell population together with instructions for preparing the enriched T cell preparation. The antibody compositions are described above and in Table 1.

In one embodiment, the kit is useful in preparing a cell preparation enriched in T cells and comprises antibodies specific for the antigens (a) CD16 and/or CD66b and/or CD11b and/or CD15; (b) CD19 and/or CD20 and/or CD21 and/or CD22 and/or CD24 and/or Ig; and (c) CD14 and/or CD36, and instructions for preparing a cell preparation enriched in T cells. Optionally, the T cell composition additionally includes antibodies to CD13, CD33, CD34, CD56, IgE, CD41 or glycophorin A.

In a specific embodiment, the kit for preparing a cell preparation enriched in T cells comprises antibodies specific for the antigens CD16, CD19 and CD36 and optionally CD56 and glycophorin A as well as instructions for the use thereof.

The following non-limiting examples are illustrative of the present invention:

### EXAMPLES

### Example 1

### Immunomagnetic Method for Evaluating Antibody Combinations

Suspensions of normal peripheral blood mononuclear cells were labelled with tetrameric antibodies and colloidal dextran iron for magnetic cell depletions. Monoclonal antibodies recognizing specific cell surface antigens were mixed with a mouse IgG₁ anti-dextran antibody (Thomas, T. E, et al. (1992), J. Immunol Methods 154:245;252) and a rat IgG₁ monoclonal antibody which recognizes the Fc portion of the mouse IgG₁ molecule (TFL-P9) (Lansdorp, P. M, and Thomas, T. E. (1990), Mol. Immunol. 27:659-666). Tetrameric antibody complexes (Lansdorp, P. M, and Thomas, T. E. (1990), Mol. Immunol. 27:659-666; U.S. Pat. No. 4,868,109 to Lansdorp) spontaneously form when mouse IgG₁ molecules (the lineage specific monoclonal antibody and anti-dextran) are mixed with P9. A proportion of these tetrameric antibody complexes are bifunctional, recognizing an antigen on the surface of the target cell on one side and dextran (part of the magnetic colloidal dextran iron) on the other. Tetrameric antibody complexes were made for all the antibodies in the lineage cocktail. FIG. 1 shows a schematic representation of magnetic cell labelling using tetrameric antibody complexes and colloidal dextran iron.

Cells were labelled for separation (1-5×10⁷ cells/ml) by incubating them with the desired combination of tetramers for 30 min on ice followed by a 30 min incubation with colloidal dextran iron (final OD450=0.6) (Molday and MacKenzie 1982, 52(3): 353-367). The cells were then passed through a magnetic filter (U.S. Pat. No. 5,514,340; inventors Lansdorp and Thomas) at 1 cm/min. The magnetically labelled cells bind to the filter and the unlabeled cells pass through. FIG. 1 shows a schematic representation of magnetic cell labeling using tetrameric antibody complexes and colloidal dextran iron.

The flow through fraction is collected and analyzed for T cell subsets.

### Example 2

### Enrichment of Resting T Cells

This example demonstrates the enrichment of resting T cells from peripheral blood mononucelar cells using the method of immunomagnetic negative selection described in Example 1. Cells were labelled with three different cocktails of tetrameric antibody complexes. All cocktails contained antibodies to CD14, CD16, CD19, CD56, glycophorin A but varied in the addition of anti-HLA-DR, CD25 or CD69. The results, shown in Table 3, demonstrate that the method of the invention highly enriches resting T cells with good recovery and efficiently eliminates activated T cells.

### Example 3

### Enrichment of αβTCR T Cells

This example demonstrates the enrichment of αβTCR+ T cells from peripheral blood mononucelar cells using the method of immunomagnetic negative selection described in Example 1. Cells were labeled with a cocktail of tetrameric antibody complexes recognizing γδTCR, CD14, CD16, CD19, CD56, and glycophorin A. The results, shown in Table 4, demonstrate that the method of the invention enriches αβTCR+ T cells to >95% purity. Contaminating γδ TCR+ T cells were less than 0.2% of the cells.

### Example 4

### Enrichment of naïve CD4+ T Cells

This example demonstrates the enrichment of naïve CD4+ T cells from peripheral blood mononucelar cells using the method of immunomagnetic negative selection described in Example 1. Cells were labeled with two different cocktails of tetrameric antibody complexes. One recognizing CD29, CD8, CD14, CD16, CD19, CD56, and glycophorin A and the other recognizing CD8, CD45RO, CD14, CD16, CD19, CD56, and glycophorin A. The results, shown in Table 5, demonstrate that the cocktail with anti-CD45RO is superior to the cocktail with anti CD29.

### Example 5

### Enrichment of Memory CD4+ T Cells

This example demonstrates the enrichment of memory CD4+ T cells from peripheral blood mononucelar cells using the method of immunomagnetic negative selection described in Example 1. Cells were labeled with a cocktail of tetrameric antibody complexes recognizing CD45RA, CD8, CD14, CD16, CD19, CD56, and glycophorin A. The results, shown in Table 6, demonstrate that the method of the invention enriches Memory CD4+ T cells to 63-72% purity with 31-99% recovery.

### Example 6

### Enrichment of CD4⁺ TH1 T Cells

This example demonstrates the enrichment of CD4⁺ TH1 T cells from peripheral blood mononucelar cells using the method of immunomagnetic negative selection described in Example 1. Cells were labeled with a cocktail of tetrameric antibody complexes recognizing CD8, CD124, CD14, CD16, CD19, CD56, and glycophorin A. The results, shown in Table 7, demonstrate that the method of the invention highly enriches CD124 negative T cells to 99% purity with 32% recovery.

### Example 7

### Method of Immunorosetting Using Ficoll

A negative selection protocol for immunorosetting cells from whole peripheral blood using Ficoll Hypaque is set out below.

1. Add 10 μL antibody composition per mL of whole peripheral blood. This antibody composition consists of tetrameric antibody complexes recognizing glycophorin A on erythrocytes and different cell surface antigens. Rosettes are formed between erythrocytes and cells expressing the target antigens.

2. Incubate 20 minutes at room temperature.

3. Dilute sample with an equal volume of phosphate buffered saline (PBS)+2% fetal calf serum (FCS) and mix gently.

4. Layer the diluted sample on top of Ficoll Hypaque or layer the Ficoll underneath the diluted sample.

5. Centrifuge for 20 minutes at 1200×g, room temperature, with the brake off. Rosetted cells sink to the pellet.

6. Remove the enriched cells from the Ficoll:plasma interface.

7. Wash enriched cells with 5-10×volume of PBS+2% FBS.

### Example 8

### T Cell Enrichment—Effect of Substituting Anti-CD14 with Anti-CD36

This example demonstrates the improved T cell enrichment (CD3⁺ T cells, CD4⁺ T cells and CD8⁺ T cells) from whole peripheral blood using the immunoresetting method described in Example 7. In this example the T cell enrichment cocktail (comprising antibodies that bind to CD14, CD16, CD19, CD56 and glycophorin A) or the CD4+ T cell enrichment cocktail (comprising antibodies that bind to CD8, CD14, CD16, CD19, CD56 and glycophorin A) or the CD8⁺ T cell enrichment cocktail (comprising antibodies to bind to CD4, CD14, CD16, CD19, CD56 and glycophorin) is modified by substituting anti-CD36 for anti-CD14. The results in Table 8 show an increase in purity of CD3⁺, CD4⁺ and CD8⁺ T cells with the antibody substitution.

### Example 9

### Enrichment of γδ TCR Positive T Cells

This example demonstrates the enrichment of γδ TCR positive T Cells from peripheral blood mononuclear cells using the method of immunomagnetic negative selection described in Example 1. Cells were labeled with a cocktail of tetrameric antibody complexes recognizing αβTCR, CD14, CD16, CD19, CD56, and glycophoril A. The results shown in Table 9, demonstrate that the method of the invention highly enriches for γδ TCR positive T cells.

### Example 10

### Enrichment of Resting CD4+ T Cells

This example demonstrates the enrichment of resting CD4+ T Cells from peripheral blood mononuclear cells using the method of immunomagnetic negative selection described in Example 1. Cells were labeled with a cocktail of tetrameric antibody complexes recognizing HLA-DR, CD25, CD8, CD14, CD16, CD19, CD56, and glycophorin A. The results shown in Table 10, demonstrate that the method of the invention enriches resting CD4+ T cells to 97% with greater than 2.5 log depletion of activated CD4+ T cells. The recovery of resting CD4+ T cells was 26%.

### Example 11

### Enrichment of CD4+ αβ T Cells

This example demonstrates the enrichment of CD4+ αβ T Cells from peripheral blood mononuclear cells using the method of immunomagnetic negative selection described in Example 1. Cells were labeled with a cocktail of tetrameric antibody complexes recognizing γδTCR, CD8, CD14, CD16, CD19, CD56, and glycophorin A. The results shown in Table 11, demonstrate that the method of the invention enriches CD4+αβ T Cells to 94% purity with 82% recovery. CD4+γδ T cells were not detectable by FACS in the enriched fraction (less than 1 in 50,000 events).

### Example 12

### Enrichment of Naive CD8+ T Cells

This example demonstrates the enrichment of Naive CD8+ T Cells from peripheral blood mononuclear cells using the method of immunomagnetic negative selection described in Example 1. Cells were labeled with a cocktail of tetrameric antibody complexes recognizing CD45RO, CD4, CD14, CD16, CD19, CD56, and glycophorin A. Naive CD8+ T cells (CD8+CD45RA+) were enriched from 14.5% to 94.4% with 28.4% recovery. Memory CD8+T cells (CD8+CD45RO+) were depleted by 2.3 log (3.7%-0.4% of total cells).

### Example 13

### Enrichment of memory CD8+ T cells

This example demonstrates the enrichment of Memory CD8+ T cells from peripheral blood mononuclear cells using the method of immunomagnetic negative selection described in Example 1. Cells were labeled with a cocktail of tetrameric antibody complexes recognizing CD45RA, CD4, CD14, CD16, CD19, CD56, and glycophorin A. Memory CD8+ T cells (CD8+CD45RO+) were enriched from 5.0% to 54.9% (10 fold) with 1.3% recovery and 4.8 log depletion of Naive CD8+ T cells (CD8+CD45RA+).

### Example 14

### Enrichment of Resting CD8+ T Cells

This example demonstrates the enrichment of resting CD8+ T Cells from peripheral blood mononuclear cells using the method of immunomagnetic negative selection described in Example 1. Cells were labeled with a cocktail of tetrameric antibody complexes recognizing HLA-DR, CD25, CD4, CD14, CD16, CD19, CD56, and glycophorin A. The results shown in Table 12, demonstrate that the method of the invention enriches resting CD8+ T cells to 97% with 1.7-3.2 log depletion of activated CD8⁺ T cells (CD8+CD25+, CD8+HLA-DR+ respectively). The recovery of resting CD8+ T cells (CD8+HLA-DR⁻ or CD25⁻) was 32%.

### Example 15

### Enrichment of CD8+ αβ T Cells

This example demonstrates the enrichment of CD8+ αβ T Cells from peripheral blood mononuclear cells using the method of immunomagnetic negative selection described in Example 1. Cells were labeled with a cocktail of tetrameric antibody complexes recognizing γδTCR, CD4, CD14, CD16, CD19, CD56, and glycophorin A. The results shown in Table 13, demonstrate that the method of the invention enriches CD8+αβ T Cells to 91% purity with 39% recovery. CD8+γδ T cells were depleted by 1.6 log.

While what is shown and described herein constitutes various preferred embodiments of the subject invention, it will be understood that various changes can be made to such embodiments without departing from the subject invention, the scope of which is defined in the appended claims.

TABLE 1
Antibody Cocktails

T Cell Enrichment
Anti-
CD16 and/or CD66b, CD11b, CD15
CD19 and/or CD20, CD21, CD22, CD24, Ig
CD14 and/or CD36
and optionally anti-CD33, CD56, IgE, CD41, glycophorin A, CD13,
CD34
CD4⁺T Cell Enrichment
Anti-
CD8
CD16 and/or CD66b, CD11b, CD15
CD19 and/or CD2O, CD21, CD22, CD24, Ig
CD14 and/or CD36
and optionally anti-CD33, CD56, IgE, CD41, glycophorin A, CD13,
CD34
CD8⁺T Cell Enrichment
Anti-
CD4
CD16 and/or CD66b, CD11b, CD15
CD19 and/or CD20, CD21, CD22, CD24, Ig
CD14 and/or CD36
and optionally anti-CD33, CD56, IgE, CD41, glycophorin A, CD13,
CD34
Resting T Cell Enrichment
Anti-
HLA-DR and/or CD25, CD69, CD27
CD16 and/or CD66b, CD11b, CD15
CD19 and/or CD20, CD21, CD22, CD24, Ig
CD14 and/or CD36
and optionally anti-CD33, CD56, IgE, CD41, glycophorin A, CD13,
CD34
γδ T Cell Enrichment
Anti-
αβTCR
CD16 and/or CD66b, CD11b, CD15
CD19 and/or CD20, CD21, CD22, CD24, Ig
CD14 and/or CD36
and optionally anti-CD33, CD56, IgE, CD41, glycophorin A, CD13,
CD34
αβT Cell Enrichment
Anti-
γδTCR
CD16 and/or CD66b, CD11b, CD15
CD19 and/or CD20, CD21, CD22, CD24, Ig
CD14 and/or CD36
and optionally anti-CD33, CD56, IgE, CD41, glycophorin A, CD13,
CD34
Naïve T Cell Enrichment
Anti-
CD45RO and/or CD29
CD16 and/or CD66b, CD11b, CD15
CD19 and/or CD20, CD21, CD22, CD24, Ig
CD14 and/or CD36
and optionally anti-CD33, CD56, IgE, CD41, glycophorin A, CD13,
CD34
Naïve CD4 + T Cell Enrichment
Anti-
CD8
CD45RO and/or CD29
CD16 and/or CD66b, CD11b, CDT5
CD19 and/or CD20, CD21, CD22, CD24, Ig
CD14 and/or CD36
and optionally anti-CD33, CD56, IgE, CD41, glycophorin A, CD13,
CD34
Memory T Cell Enrichment
Anti-
CD45RA
CD16 and/or CD66b, CD11b, CD15
CD19 and/or CD20, CD21, CD22, CD24, Ig
CD14 and/or CD36
and optionally anti-CD33, CD56, IgE, CD41, glycophorin A, CD13,
CD34
Memory CD4 + T Cell Enrichment
Anti-
CD8
CD45RA
CD16 and/or CD66b, CD11b, CD15
CD19 and/or CD20, CD21, CD22, CD24, Ig
CD14 and/or CD36
and optionally anti-CD33, CD56, IgE, CD41, glycophorin A, CD13,
CD34
Resting CD4+ T Cell Enrichment
Anti-
CD8
HLA-DR and/or CD25, CD69, CD27
CD16 and/or CD66b, CD11b, CD15
CD19 and/or CD20, CD21, CD22, CD24, Ig
CD14 and/or CD36
and optionally anti-CD33, CD56, IgE, CD41, glycophorin A, CD13,
CD34
CD4+ αβT Cell Enrichment
Anti-
γδTCR
CD8
CD16 and/or CD66b, CD11b, CD15
CD19 and/or CD20, CD21, CD22, CD24, Ig
CD14 and/or CD36
and optionally anti-CD33, CD56, IgE, CD41, glycophorin A, CD13,
CD34
TH1 CD4+ T Cell Enrichment
Anti-
CD8
CD124
CD16 and/or CD66b, CD11b, CD15
CD19 and/or CD20, CD21, CD22, CD24, Ig
CD14 and/or CD36
and optionally anti-CD33, CD56, IgE, CD41, glycophorin A, CD13,
CD34
TH2 CD4+ T Cell Enrichment
Anti-
CD8
CCR5
CD16 and/or CD66b, CD11b, CD15
CD19 and/or CD20, CD21, CD22, CD24, Ig
CD14 and/or CD36
and optionally anti-CD33, CD56, IgE, CD41, glycophorin A, CD13,
CD34
Native CD8+ T Cell Enrichment
Anti-
CD4
CD45RO and/or CD29
CD16 and/or CD66b, CD11b, CD15
CD19 and/or CD20, CD21, CD22, CD24, Ig
CD14 and/or CD36
and optionally anti-CD33, CD56, IgE, CD41, glycophorin A, CD13,
CD34
Memory CD8+ T Cell Enrichment
Anti-
CD4
CD45RA
CD16 and/or CD66b, CD11b, CD15
CD19 and/or CD20, CD21, CD22, CD24, Ig
CD14 and/or CD36
and optionally anti-CD33, CD56, IgE, CD41, glycophorin A, CD13,
CD34
Resting CD8+ T Cell Enrichment
Anti-
CD4
HLA-DR and/or CD25, CD69, CD27
CD16 and/or CD66b, CDUb, CD15
CD19 and/or CD20, CD21, CD22, CD24, Ig
CD14 and/or CD36
and optionally anti-CD33, CD56, IgE, CD41, glycophorin A, CD13,
CD34
CD8+ αβT Cell Enrichment
Anti-
γδTCR
CD4
CD16 and/or CD66b, CD11b, CD15
CD19 and/or CD2O, CD21, CD22, CD24, Ig
CD14 and/or CD36
and optionally anti-CD33, CD56, IgE, CD41, glycophorin A, CD13,
CD34

TABLE 2
Antibodies used in Cell Separation

| Antigen | Antibody | Source |
|---|---|---|
| CCR5 | BLR-7 | R&D, Minneapolis, MN |
| CD4 | 13B8.2 | Becton Dickinson Immunocytometry, |
| | | Mountain View, Calif. |
| CD8 | B911 | Becton Dickinson Immunocytometry, |
| | | Mountain View, Calif. |
| | OKT3 | BioDesigns |
| CD10 | ALB1 | IMMUNOTECH, Marseille, France |
| CD11b | ICRF44 | Pharmingen, San Diego, CA |
| CD13 | SJ1DF | IMMUNOTECH, Marseille, France |
| | WM-47 | Dako, Carpinteria, CA |
| | WM-15 | Becton Dickinson Immunocytometry, |
| | | Mountain View, Calif. |
| CD14 | MEM 15 | Exbio, Praha, Czech Republic |
| | MEM 18 | |
| CD15 | DU-HL60-3 | Sigma, St. Louis, MO |
| CD16 | MEM 154 | Exbio, Praha, Czech Republic |
| | 3G8 | IMMUNOTECH, Marseille, France |
| | NKP15 | Becton Dickinson Immunocytornetry, |
| | | Mountain View, Calif. |
| CD19 | J4.119 | IMMUNOTECH, Marseille, France |
| | 4G7 | Becton Dickinson Immunocytometry, |
| | | Mountain View, Calif. |
| | HD37 | Dako, Carpinteria, CA |
| CD20 | MEM97 | Exbio, Praha, Czech Republic |
| | L27 | Becton Dickinson Immunocytometry, |
| | | Mountain View, Calif. |
| CD21 | B-Ly4 | Pharmingen, San Diego, CA |
| CD22 | H1B22 | Pharmingen, San Diego, CA |
| CD24 | 32D12 | Dr. Steinar Funderud, Institute |
| | | for Cancer |
| | | Research, Dept. of Immunology, Oslo, |
| | | Norway |
| | ALB9 | IMMUNOTECH, Marseille, France |
| CD25 | 3G10 | Caltaq, Burlingame, CA |
| CD27 | 1A4CD27 | IMMUNOTECH, Marseille, France |
| CD29 | Lia1.2 | IMMUNOTECH, Marseille, France |
| CD33 | D3HL60.251 | IMMUNOTECH, Marseille, France |
| CD34 | 581 | IMMUNOTECH, Marseille, France |
| CD36 | FA6.152 | IMMUNOTECH, Marseille, France |
| | IVC7 | CLB, Central Laboratory of the |
| | | Netherlands, Red Cross Blood |
| | | Transfusion Service |
| CD38 | T16 | IMMUNOTECH, Marseille, France |
| CD41 | P11.64 | Kaplan, 5th International Workshop |
| | | on Human Leukocyte |
| | | Differentiation Antigens |
| | SZ22 | IMMUNOTECH, Marseille, France |
| CD42a | Bebl | Becton Dickinson Immunocytometry, |
| | | Mountain View, Calif. |
| CD45RA | 8D2.2 | Craig et al. 1994, StemCell |
| | | Technologies, Vancouver, Canada |
| | L48 | Becton Dickinson Immunocytometry, |
| | | Mountain View, Calif. |
| CD45RO | UCHL1 | Dako, Carpinteria, CA |
| CD56 | T199 | IMMUNOTECH, Marseille, France |
| | MY31 | Becton Dickinson Immunocytometry, |
| | | Mountain View, Calif. |
| CD66e | CLB/gran10 | CLB, Central Laboratory of the Netherlands, |
| | | Red Cross Blood |
| | | Transfusion Service |
| CD66b | B13.9 | CLB, Central Laboratory of the Netherlands, |
| | | Red Cross Blood |
| | | Transfusion Service |
| | 80H3 | IMMUNOTECH, Marseille, France |
| CD69 | L78 | BD Biosciences, San Jose, CA |
| CD71 | My29 | Zymed Laboratories, San Francisco, CA |
| CD124 | S456C9 | IMMUNOTECH, Marseille, France |
| HLADRR | IMMU357.12 | IMMUNOTECH, Marseille, France |
| IgA1 | NiF2 | IMMUNOTECH, Marseille, France |
| IgE | G7-18 | Pharmingen, San Diego, CA |
| IgG | 8A4 | IMMUNOTECH, Marseille, France |
| TCRαβ | WT31 | BD Biosciences, San Jose, CA |
| TCR γδ | Immu510 | IMMUNOTECH, Marseille, France |

TABLE 3
Enrichment of Resting T Cells

| | % Purity | Residual | % Recovery |
|---|---|---|---|
| Coctail | Resting T cells | activated T cells | resting T cells |
| HLA-DR, CD14, CD16, CD19, | 8.6% | 0.09% | 86% of |
| CD56, glycophorin A | CD3⁺CD25⁻cells | CD3⁺CD25⁺cells | CD3⁺DR⁻cells |
| CD25, CD14, CD16, CD19, | 90.4% | 1.0% | 68% of |
| CD56, glycophorin A | CD3⁺DR⁻cells | CD3⁺DR⁺cells | CD3⁺CD25⁻cells |
| CD69, CD14, CD16, CD19, | 98.6% | 0.36% | 67% of |
| CD56, glycophorin A | CD3⁺DR⁻cells | CD3⁺DR⁺cells | CD3⁺CD69⁻cells |

TABLE 4
Enrichment of αβTCR T Cells

| | % Purity | % Recovery |
|---|---|---|
| Sample | αβTCR T cells | αβTCR T cells |
| 1 | 94.8 | 37 |
| 2 | 98.9 | 100 |

TABLE 5
Enrichment of Naïve CD4+ T Cells

| | % purity | % Recovery |
|---|---|---|
| | CD4⁺CD45RA⁺CD45RO⁻ | CD4⁺CD45RA⁺CD45RO⁻ |
| Cocktail | cells | cells |
| CD29, CD8, | 73 | 28 |
| CD14, CD16, | | |
| CD19, CD56, | | |
| glycophorin A | | |
| CD45RO, | 86 | 58 |
| CD8, CD14, | | |
| CD16, CD19, | | |
| CD56, | | |
| glycophorin A | | |

TABLE 6
Enrichment of Memory CD4+ Cells

| Sam- | % Purity | % Recovery |
|---|---|---|
| ple | CD4⁺CD45RO⁺CD45RA⁻cells | CD4⁺CD45RO⁺CD45RA⁻cells |
| 1 | 63 | 99 |
| 2 | 72 | 31 |

TABLE 7
Enrichment of CD4+ TH1 T Cells

| % Purity | % Recovery |
|---|---|
| CD4⁺CD124⁻T cells | CD4⁺CD124⁻T cells |
| 99 | 32 |

TABLE 8
T Cell Enrichment-Immunorosetting Using Ficoll

| A: T cell enrichment | | | |
|---|---|---|---|
| | | Cocktail with CD14 | Cocktail with CD36 |
| | n | ±1SD | ±1SD |
| Purity | 3 | 80 ± 10 | 94 ± 5 |
| Recovery | 3 | 56 ± 12 | 42 ± 10 |
| B: CD4 T cell enrichment | | | |
| | | cocktail with | |
| | n | CD14 | cocktail with CD36 |
| start | 2 | 44, 69 | 64, 85 |
| enriched | 2 | 21, 35 | 13, 27 |
| C: CD8 T cell enrichment | | | |
| | | cocktail with | |
| | n | CD14 | cocktail with CD36 |
| start | 1 | 21 | 334 |
| enriched | 1 | 35 | 14 |

TABLE 9
Enrichment of γδ TCR Positive T Cells

| | Start | Start | | | |
|---|---|---|---|---|---|
| | % | % | | | |
| | γδ T | αβ T | Enriched | Enriched | % Recovery |
| | cells | cells | % γδ T cells | % αβ T cells | γδ T cells |
| % Total Cells | 0.4 | 35.5 | 7.6 | 0.4 | 11.3 |
| % | 0.7 | ND | 45.3 | ND | 11.3 |
| CD4⁺/CD8⁺ | | | | | |
| lymphocytes | | | | | |

TABLE 10
Enrichment of Resting CD4+ T Cells

| | % CD4+ | | |
|---|---|---|---|
| | HLA-DR⁻ | | |
| | and/or CD25⁻ | % CD4 + HLA − DR⁺ | % CD4 + CD25⁺ |
| Start | 24.7 | 1.07 | 0.15 |
| Enriched | 97.3 | 0.05 | 0.004 |

TABLE 11
Enrichment of CD4+ αβ T Cells

| Start | Enriched | % Recovery |
|---|---|---|
| % CD4+αβ T cells | % CD4+ αβ T cells | CD4+ αβ T cells |
| 24.2 | 94.2 | 82.3 |

TABLE 12
Enrichment of Resting CD8+ T cells

| | % CD8+ | | |
|---|---|---|---|
| | HLA-DR- and/ | | |
| | or CD25− | % CD8+ HLA-DR+ | % CD8+ CD25⁺ |
| Start | 17 | 0.55 | 0.02 |
| Enriched | 97 | <0.006 | 0.006 |

TABLE 13
Enrichment of CD8+ αβ T Cells

| Start | Enriched | % Recovery |
|---|---|---|
| % CD8+ αβ T cells | % CD8+ αβ T cells | CD8+ αβ T cells |
| 17 | 91 | 39 |

We claim:

1. An antibody composition for enriching for T cells comprising antibodies specific for the antigens
(a) CD16;
(b) CD19; and
(c) CD36.

2. An antibody composition according to claim 1 for enriching for CD4⁺ T cells comprising antibodies specific for the antigens
(a) CD16;
(b) CD19;
(c) CD36; and
(d) CD8.

3. An antibody composition according to claim 1 for enriching for CD8⁺ T cells comprising antibodies specific for the antigens
(a) CD16;
(b) CD19;
(c) CD36; and
(d) CD4.

4. An antibody composition according to claim 1 further comprising antibodies specific for the antigen CD56.

5. An antibody composition according to claim 1 further comprising antibodies specific for the antigen glycophorin A.

6. An antibody composition for enriching for naive CD4⁺ T cells comprising antibodies specific for the antigens:
(a) CD16;
(b) CD19;
(c) CD14;
(d) CD8; and
(e) CD45RO.

7. An antibody composition according to claim 6 further comprising antibodies specific for the antigen CD56 or glycophorin A.

8. An antibody composition for enriching for resting T cells comprising antibodies specific for the antigens:
(a) CD16 and/or CD66b and/or CD11b and/or CD15;
(b) CD19 and/or CD20 and/or CD21 and/or CD22 and/or CD24 and/or Ig;
(c) CD14 and/or CD36; and
(d) HLA-DR and/or CD25 and/or CD69 and/or CD27.

9. An antibody composition for enriching for γδ T cells comprising antibodies specific for the antigens:
(a) CD16 and/or CD66b and/or CD11b and/or CD15;
(b) CD19 and/or CD20 and/or CD21 and/or CD22 and/or CD24 and/or Ig;
(c) CD14 and/or CD36; and
(d) αβ TCR.

10. An antibody composition for enriching for αβ T cells comprising antibodies specific for the antigens:
(a) CD16 and/or CD66b and/or CD11b and/or CD15;
(b) CD19 and/or CD20 and/or CD21 and/or CD22 and/or CD24 and/or Ig;
(c) CD14 and/or CD36; and
(d) γδ TCR.

11. An antibody composition for enriching for memory T cells comprising antibodies specific for the antigens:
(a) CD16 and/or CD66b and/or CD11b and/or CD15;
(b) CD19 and/or CD20 and/or CD21 and/or CD22 and/or CD24 and/or Ig;
(c) CD14 and/or CD36; and
(d) CD45RA.

12. An antibody composition for enriching for naive T cells comprising antibodies specific for the antigens:
(a) CD16 and/or CD66b and/or CD11b and/or CD15;
(b) CD19 and/or CD20 and/or CD21 and/or CD22 and/or CD24 and/or Ig;
(c) CD14 and/or CD36; and
(d) CD45RO and/or CD29.

13. An antibody composition according to claim 11 for enriching for memory CD4⁺ T cells comprising antibodies specific for the antigens:
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) CD8 and
(e) CD45RA.

14. An antibody composition according to claim 8 for enriching for resting CD4⁺ T cells comprising antibodies specific for the antigens:
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) CD8 and
(e) HLA-DR and/or CD25 and/or CD69 and/or CD27.

15. An antibody composition according to claim 10 for enriching for CD4⁺ αβ T cells comprising antibodies specific for the antigens:
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) γδ TCR; and
(e) CD8.

16. An antibody composition for enriching for TH₁ CD4⁺ T cells comprising antibodies specific for the antigens:
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) CD8; and
(e) CD124.

17. An antibody composition for enriching for TH2 CD4⁺ T cells comprising antibodies specific for the antigens:
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) CD8; and
(e) CCR5.

18. An antibody composition according to claim 12 for enriching for naive CD8⁺ T cells comprising antibodies specific for the antigens:
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) CD4; and
(e) CD45RO and/or CD29.

19. An antibody composition according to claim 11 for enriching for memory CD8⁺ T cells comprising antibodies specific for the antigens:
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) CD4; and
(e) CD45RA.

20. An antibody composition according to claim 8 for enriching for resting CD8⁺ T cells comprising antibodies specific for the antigens:
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) CD4; and
(e) HLA-DR and/or CD25 and/or CD69 and/or CD27.

21. An antibody composition according to claim 10 for enriching for CD8⁺ αβ T cells comprising antibodies specific for the antigens:
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) γδ TCR; and
(e) CD4.

22. An antibody composition according to claim 1, wherein the antibodies are monoclonal antibodies.

23. An antibody composition according to claim 22 wherein the antibodies are labelled with a marker or they are directly or indirectly conjugated to a matrix.

24. An antibody composition according to claim 22 wherein the antibodies are labelled with biotin or a fluorochrome.

25. An antibody composition according to claim 24 wherein the matrix is magnetic beads, a panning surface, dense particles for density centrifugation, an adsorption column, or an adsorption membrane.

26. An antibody composition according to claim 22 wherein each of the monoclonal antibodies is incorporated in a tetrameric antibody complex which comprises a first monoclonal antibody of a first animal species from the antibody composition according to claim 22, and a second monoclonal antibody of the first animal species which is capable of binding to at least one antigen on the surface of a matrix, which have been conjugated to form a cyclic tetramer with two monoclonal antibodies of a second animal species directed against the Fc-fragments of the antibodies of the first animal species.

27. A negative selection process for enriching and recovering T cells in a sample comprising
(1) reacting the sample with an antibody composition containing antibodies capable of binding to the antigens
(a) CD16;
(b) CD19; and
(c) CD36, under conditions so that conjugates are formed between the antibodies and cells in the sample containing the antigens
(a) CD16;
(b) CD19; and
(c) CD36, on their surfaces;
(2) removing the conjugates; and
(3) recovering a cell preparation which is enriched in T cells.

28. A negative selection process according to claim 27 for enriching and recovering CD4⁺ T cells wherein the antibody composition further comprises antibodies capable of binding to the antigen CD8.

29. A negative selection process according to claim 27 for enriching and recovering CD8⁺ T cells wherein the antibody composition further comprises antibodies capable of binding to the antigen CD4.

30. A negative selection process according to claim 27 further comprising antibody specific for the antigen CD56.

31. A negative selection process according to claim 27 further comprising antibody specific for the antigen glycophorin A.

32. A negative selection process for enriching for naive CD4⁺ T cells comprising
(1) reacting the sample with an antibody composition containing antibodies capable of binding to the antigens
(a) CD16;
(b) CD19;
(c) CD14;
(d) CD8; and
(e) CD45RO, under conditions so that conjugates are formed between the antibodies and cells in the sample containing the antigens
(a) CD16;
(b) CD19;
(c) CD14;
(d) CD8; and
(e) CD45RO, on their surfaces;
(2) removing the conjugates; and
(3) recovering a cell preparation which is enriched in naïve CD4⁺ T cells.

33. A negative selection process according to claim 32 further comprising antibodies specific for the antigen CD56 or glycophorin A.

34. A negative selection process for enriching and recovering γδ T cells in a sample comprising
(1) reacting the sample with an antibody composition containing antibodies capable of binding to the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36; and
(d) αβ TCR, under conditions so that conjugates are formed between the antibodies and cells in the sample containing the antigens
(a) CD16 and/or CD66b and/or CD11b and/or CD15;
(b) CD19 and/or CD20 and/or CD21 and/or CD22 and/or CD24 and/or Ig; and
(c) CD14 and/or CD36 and
(d) αβ TCR, on their surfaces;
(2) removing the conjugates; and
(3)recovering a cell preparation which is enriched in γδ T cells.

35. A negative selection process for enriching and recovering αβ T cells in a sample comprising
(1) reacting the sample with an antibody composition containing antibodies capable of binding to the antigens
(a) CD16 and/or CD66b and/or CD11b and/or CD15;
(b) CD19 and/or CD20 and/or CD21 and/or CD22 and/or CD24 and/or Ig; and
(c) CD14 and/or CD36; and
(d) γδ TCR, under conditions so that conjugates are formed between the antibodies and cells in the sample containing the antigens
(a) CD16 and/or CD66b and/or CD11b and/or CD15;
(b) CD19 and/or CD20 and/or CD21 and/or CD22 and/or CD24 and/or Ig; and
(c) CD14 and/or CD36 and
(d) γδ TCR, on their surfaces;
(2) removing the conjugates; and
(3) recovering a cell preparation which is enriched in αβ T cells.

36. A negative selection process for enriching and recovering memory T cells in a sample comprising
(1) reacting the sample with an antibody composition containing antibodies capable of binding to the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36; and
(d) CD45RA, under conditions so that conjugates are formed between the antibodies and cells in the sample containing the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36; and
(d) CD45RA;
(2) removing the conjugates; and
(3) recovering a cell preparation which is enriched in memory T cells.

37. A negative selection process for enriching and recovering naïve T cells in a sample comprising
(1) reacting the sample with an antibody composition containing antibodies capable of binding to the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36; and
(d) CD45RO and/or CD29, under conditions so that conjugates are formed between the antibodies and cells in the sample containing the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36; and
(d) CD45RO and/or CD29;
(2) removing the conjugates; and
(3) recovering a cell preparation which is enriched in naïve T cells.

38. A negative selection process for enriching and recovering resting T cells in a sample comprising
(1) reacting the sample with an antibody composition containing antibodies capable of binding to the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) HLA-DR and/or CD25 and/or CD69 and/or CD27 CD8, under conditions so that conjugates are formed between the antibodies and cells in the sample containing the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) HLA-DR and/or CD25 and/or CD69 and/or CD27, on their surfaces;
(2) removing the conjugates; and
(3) recovering a cell preparation which is enriched in naive CD4⁺ T cells.

39. A negative selection process according to claim 36 for enriching and recovering memory CD4⁺ T cells in a sample comprising
(1) reacting the sample with an antibody composition containing antibodies capable of binding to the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) CD8 and
(e) CD45RA, under conditions so that conjugates are formed between the antibodies and cells in the sample containing the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) CD8 and
(e) CD45RA, on their surfaces;
(2) removing the conjugates; and
(3) recovering a cell preparation which is enriched in memory CD4⁺ T cells.

40. A negative selection process according to claim 38 for enriching and recovering resting CD4⁺ T cells in a sample comprising
(1) reacting the sample with an antibody composition containing antibodies capable of binding to the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) CD8 and
(e) HLA-DR and/or CD25 and/or CD69 and/or CD27, under conditions so that conjugates are formed between the antibodies and cells in the sample containing the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) CD8 and
(e) HLA-DR and/or CD25 and/or CD69 and/or CD27, on their surfaces;
(2) removing the conjugates; and
(3)recovering a cell preparation which is enriched in resting CD4⁺ T cells.

41. A negative selection process according to claim 35 for enriching and recovering CD4⁺ αβ T cells in a sample comprising
(1) reacting the sample with an antibody composition containing antibodies capable of binding to the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) γδ TCR; and
(e) CD8, under conditions so that conjugates are formed between the antibodies and cells in the sample containing the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) γδ TCR; and
(e) CD8, on their surfaces;
(2) removing the conjugates; and
(3)recovering a cell preparation which is enriched in CD4⁺ αβ T cells.

42. A negative selection process for enriching and recovering TH1 CD4⁺ T cells in a sample comprising
(1) reacting the sample with an antibody composition containing antibodies capable of binding to the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) CD8; and
(e) CD124, under conditions so that conjugates are formed between the antibodies and cells in the sample containing the antigens
(a) CD16 and/or CD66b and/or CD11b and/or CD15;
(b) CD19 and/or CD20 and/or CD21 and/or CD22 and/or CD24 and/or Ig; and
(c) CD14 and/or CD36;
(d) CD8; and
(e) CD124, on their surfaces;
(2) removing the conjugates;
(3) recovering a cell preparation which is enriched in TH1 CD4⁺ T cells.

43. A negative selection process for enriching and recovering TH2 CD4⁺ T cells in a sample comprising
(1) reacting the sample with an antibody composition containing antibodies capable of binding to the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) CD8; and
(e) CCR5, under conditions so that conjugates are formed between the antibodies and cells in the sample containing the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) CD8; and
(e) CCR5, on their surfaces;
(2) removing the conjugates; and
(3) recovering a cell preparation which is enriched in TH2 CD4⁺ T cells.

44. A negative selection process according to claim 37 for enriching and recovering naïve CD8⁺ T cells in a sample comprising
(1) reacting the sample with an antibody composition containing antibodies capable of binding to the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) CD4; and
(e) CD45RO and/or CD29, under conditions so that conjugates are formed between the antibodies and cells in the sample containing the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) CD4; and
(e) CD45RO and/or CD29, on their surfaces;
(2) removing the conjugates; and
(3) recovering a cell preparation which is enriched in naive CD8⁺ T cells.

45. A negative selection process according to claim 36 for enriching and recovering memory CD8⁺ T cells in a sample comprising
(1) reacting the sample with an antibody composition containing antibodies capable of binding to the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) CD4; and
(e) CD45RA, under conditions so that conjugates are formed between the antibodies and cells in the sample containing the antigens
(a) CD16 and/or CD66b and/or CD11b and/or CD15;
(b) CD19 and/or CD20 and/or CD21 and/or CD22 and/or CD24 and/or Ig; and
(c) CD14 and/or CD36;
(d) CD4; and
(e) CD45RA, on their surfaces;
(2) removing the conjugates;
(3) recovering a cell preparation which is enriched in memory CD8⁺ T cells.

46. A negative selection process according to claim 38 for enriching and recovering resting CD8⁺ T cells in a sample comprising
(1) reacting the sample with an antibody composition containing antibodies capable of binding to the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) CD4; and
(e) HLA-DR and/or CD25 and/or CD69 and/or CD27, under conditions so that conjugates are formed between the antibodies and cells in the sample containing the antigens
(a) CD16 and/or CD66b and/or CD11b and/or CD15;
(b) CD19 and/or CD20 and/or CD21 and/or CD22 and/or CD24 and/or Ig; and
(c) CD14 and/or CD36;
(d) CD4; and
(e) HLA-DR and/or CD25 and/or CD69 and/or CD27, on their surfaces;
(2) removing the conjugates;
(3) recovering a cell preparation which is enriched in resting CD8⁺ T cells.

47. A negative selection process according to claim 35 for enriching and recovering CD8⁺ αβ T cells in a sample comprising
(1) reacting the sample with an antibody composition containing antibodies capable of binding to the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) γδ TCR; and
(e) CD4, under conditions so that conjugates are formed between the antibodies and cells in the sample containing the antigens
(a) CD16 and/or CD66b and/or CD11b and/or CD15;
(b) CD19 and/or CD20 and/or CD21 and/or CD22 and/or CD24 and/or Ig;
(c) CD14 and/or CD36;
(d) γδ TCR; and
(e) CD4, on their surfaces;
(2) removing the conjugates; and
(3) recovering a cell preparation which is enriched in CD8⁺ αβ T cells.

48. A process according to claim 27, wherein the antibodies in the antibody composition are monoclonal antibodies.

49. A process according to claim 48, wherein the antibodies in the antibody composition are labelled with a marker or they are conjugated to a matrix.

50. A process according to claim 48, wherein the antibodies in the antibody composition are labelled with biotin or a fluorochrome.

51. A process according to claim 48, wherein the matrix is magnetic beads, a panning surface, dense particles for density centrifugation, an adsorption column, or an adsorption membrane.

52. A process according to claim 48, wherein each of the monoclonal antibodies in the antibody composition is incorporated in a tetrameric antibody complex which comprises a first monoclonal antibody of a first animal species from the antibody composition according to claim 15, and a second monoclonal antibody of the first animal species which is capable of binding to at least one antigen on the surface of a matrix, which have been conjugated to form a cyclic tetramer with two monoclonal antibodies of a second animal species directed against the Fc-fragments of the antibodies of the first animal species.

53. A kit for preparing a cell preparation enriched in T cells comprising antibodies specific for the antigens CD16, CD19 and CD36 and instructions for its use in preparing the enriched T cell preparation.
